(19) United States
(12) Patent Application Publication
(10) Pub. No.: US 2024/0245660 A1
(43) Pub. Date: Jul. 25, 2024

(54) METHODS OF TREATING GASTROINTESTINAL STROMAL TUMORS

(71) Applicant: Deciphera Pharmaceuticals, LLC, Waltham, MA (US)

(72) Inventors: Kam M. Sprott, Waltham, MA (US); Matthew L. Sherman, Newton, MA (US); Rodrigo Ruiz Soto, Waltham, MA (US)

(21) Appl. No.: 18/389,888

(22) Filed: Dec. 20, 2023

Related U.S. Application Data
(60) Provisional application No. 63/515,898, filed on Jul. 27, 2023.
(60) Provisional application No. 63/505,720, filed on Jun. 2, 2023.
(60) Provisional application No. 63/493,821, filed on Apr. 3, 2023.
(60) Provisional application No. 63/481,093, filed on Jan. 23, 2023.
(60) Provisional application No. 63/478,736, filed on Jan. 6, 2023.
(60) Provisional application No. 63/435,137, filed on Dec. 23, 2022.

(51) Int. Cl. A61K 31/4375; A61P 35/00

(52) U.S. Cl. CPC A61K 31/4375; A61P 35/00

(57) ABSTRACT

The present disclosure relates to methods of treating gastrointestinal stromal tumors to a subject in need thereof, comprising administering to the subject a therapeutically effective amount of ripretinib or a pharmaceutically acceptable salt thereof.

### CROSS-REFERENCE

This application claims priority to U.S. Provisional Application No. 63/435,137 filed Dec. 23, 2022, U.S. Provisional Application No. 63/478,736 filed Jan. 6, 2023, U.S. Provisional Application No. 63/481,093 filed Jan. 23, 2023, U.S. Provisional Application No. 63/493,821 filed Apr. 3, 2023, U.S. Provisional Application No. 63/505,720 filed Jun. 2, 2023, and U.S. Provisional Application No. 63/515,898 filed Jul. 27, 2023.

### BACKGROUND

Gastrointestinal stromal tumors (GIST) comprise less than 1% of all gastrointestinal (GI) tumors, but constitute the most common mesenchymal tumors and soft tissue sarcomas of the GI tract. They occur anywhere along the GI tract but are found most often in the stomach (60%) or small intestine (30%) and less frequently in the rectum, colon, or mesentery. In the United States, around 3300 to 6000 new cases of GIST are diagnosed each year. The vast majority of cases are sporadic, and older age is a recognized risk factor. Mutations in KIT and platelet-derived growth factor receptor-alpha (PDGFRA) are found in over 80% of all primary GISTs. Alterations in neurofibromatosis type 1 gene (NF1) and succinate dehydrogenase (SDH) complex (SDHC) genes as well as altered methylation of SDHC promoter have been described as oncogenic drivers in GIST without activating mutations in KIT or PDGFRA, and they have been linked to familial and heritable syndromes (NF1 and Carney-Stratakis syndrome).

Despite a wide variation in tumor size, location, and histologic subtypes (spindle cell, epithelioid cells, and mixed type), approximately 85% of all GISTs share oncogenic mutations in 1 of 2 receptor tyrosine kinases (TKs): KIT or PDGFRA. Constitutive activation of either of these TKs plays a central role in the oncogenic behavior of GIST. The early characterization of GIST mutational status is important in both the localized and metastatic settings to identify imatinib-resistant mutations (such as some primary KIT exon 17 mutations or PDGFRA D842V) or mutations that require a higher dose of imatinib. Patients with GIST lacking KIT or PDGFRA mutations usually do not benefit from imatinib, and standard treatment algorithms mostly do not apply. However, other mutations may be present in these patients, with the largest group represented by SDH-deficiency frequently associated with Carney or Carney-Stratakis-Syndrome. Other subtypes have mutations in NF1 (usually associated with neurofibromatosis type I) or in BRAF or KRAS. Very recently, casuistic cases of GIST-like tumors harboring NTRK translocations have further expanded the spectrum of molecular subtypes.

In the pre-tyrosine kinase inhibitor (TKI) era, GISTs (often categorized as gastric leiomyosarcomas or leiomyoblastomas) were treated within the subtype of agnostic sarcoma trials and lacked an effective systemic therapy. However, a deeper understanding of the molecular pathogenesis and driving role of the protooncogenes KIT and PDGFRA has transformed the treatment of both localized and metastatic diseases. Localized and resectable tumors are treated surgically which remains the mainstay of curative therapy for localized disease. Resected high-risk GIST is typically treated with adjuvant imatinib, whereas low-risk GIST is managed with surgery alone. Intermediate-risk GIST is managed on a per-case basis. In an advanced/metastatic setting, imatinib 400 mg daily is approved, with dose escalation to 800 mg at the time of progression and has been shown to yield dramatic results in disease control. Imatinib-refractory patients are treated with sunitinib as a second-line therapy and regorafenib as third-line therapy on resistance or intolerance to sunitinib.

At diagnosis, a mutation in the KIT gene occurs in 80% of GISTs and is usually found in exon 11, and less commonly in exon 9. Both mechanisms cause ligand-independent receptor activation, which leads to uncontrolled cell growth and transformation. Primary mutations affect a loss-of-function mutation in the JM domain and lead to a shift in equilibrium toward a Type I active or on-state conformation of KIT and away from a Type II inactive or off-state conformation of KIT. Exon 11 primary mutations are the most commonly seen in GISTs (around 70% of cases) and derive significant benefit from treatment with imatinib in both the adjuvant and metastatic settings, achieving a 2-year relapse-free survival of ˜90% in the adjuvant setting, and a median event-free survival just under 2 years in the metastatic setting. Primary mutations (in treatment-naïve patients) in exon 9 affect the extracellular domain of KIT, mimicking conformational changes induced by ligand binding and triggering KIT receptor homodimerization. This dimerization leads to the activation of specific intracellular signaling pathways which can lead to cancer cell proliferation, survival, and resistance. Although less common than exon 11 mutations, exon 9 mutations (10%-15% of newly diagnosed cases) are most commonly seen in GISTs arising from the small intestine. Unlike exon 11 mutations, they benefit less from imatinib in both the adjuvant and metastatic settings.

Despite significant improvement in outcomes compared with those in the pre-mutation-driven/TKI therapy era, response to imatinib is not experienced by all patients, and most patients with GIST will ultimately develop resistance to imatinib, most commonly due to the development of secondary mutations in KIT. Secondary resistance mutations usually arise in the catalytic domain of the kinase: 1) at the switch pocket, which typically occur in KIT exons 13 and 14 or PDGFRA exons 14 and 15 and sterically disrupt drug binding or conformationally activate KIT, and 2) in the activation loop switch encoded by KIT exons 17 and 18 and PDGFRA 18. Activation loop mutations act by shifting the kinase into an activated Type I or on-state conformation that is less amenable to drug binding by any of the approved Type II TKIs. Although uncommon in primary GIST (1%-2% of newly diagnosed cases), mutations in exons 13, 14 and 17 are often responsible for acquired imatinib resistance, with exon 17 mutations alone accounting for as many as 50% of the acquired resistance cases to imatinib, and later to sunitinib. A need exists for a TKI that can inhibit clinically relevant KIT and PDGFRA mutations.

### SUMMARY

The present disclosure provides, in an embodiment, methods of treating gastrointestinal stromal tumors to a subject in need thereof, comprising administering to the subject a therapeutically effective amount of ripretinib or a pharmaceutically acceptable salt thereof.

Disclosed herein, in an embodiment, is a method of treating a patient suffering from an advanced gastrointestinal stromal tumor, where the patient has a) a KIT exon 17 and/or a KIT exon 18 mutation and/or b) a KIT exon 11 mutation, comprising: administering to the patient 150 mg of a compound represented by:

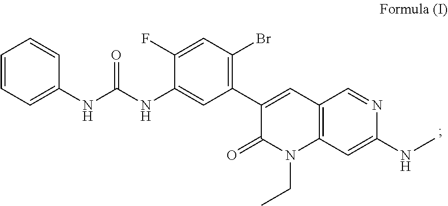 [SMILES: CCn1c(=O)c(-c2cc(NC(=O)Nc3ccccc3)c(F)cc2Br)cc2cnc(NC)cc21]

daily or twice daily, and where before the administration of the compound, the patient has progressed on or was intolerant to imatinib; and wherein upon administration of the compound the patient achieves significantly more progression free survival time as determined by mRECIST1.1, and significantly more overall survival time, as compared to a patient with a KIT exon 17 and/or a KIT exon 18 mutation and/or a KIT exon 11 mutation suffering from an advanced gastrointestinal stromal tumor who has progressed on or was intolerant to imatinib and was then administered 50 mg sunitinib once daily.

In another embodiment, described herein is a method of treating a patient suffering from an advanced gastrointestinal stromal tumor who has a KIT exon 17 and/or a KIT exon 18 mutation, does not have KIT exon 13 and/or 14 mutation, and does not have a KIT exon 9 mutation, and where the patient has progressed on or was intolerant to imatinib, comprising administering to the patient 150 mg of a compound represented by:

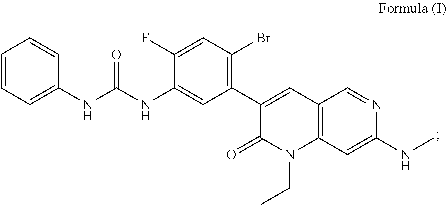 [SMILES: CCn1c(=O)c(-c2cc(NC(=O)Nc3ccccc3)c(F)cc2Br)cc2cnc(NC)cc21]

daily or twice daily.

In another embodiment, described herein is a method of treating a patient suffering from an advanced gastrointestinal stromal tumor where the patient has progressed on or was intolerant to imatinib, and the patient's circulating tumor DNA has a KIT exon 17 and/or a KIT exon 18 mutation and does not have a KIT exon 9 mutation, comprising administering to the patient 150 mg of a compound represented by:

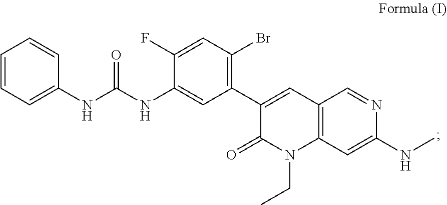 [SMILES: CCn1c(=O)c(-c2cc(NC(=O)Nc3ccccc3)c(F)cc2Br)cc2cnc(NC)cc21]

daily or twice daily.

In another embodiment, described herein is a method of treating an advanced gastrointestinal stromal tumor where the patient has progressed on or was intolerant to imatinib in a patient in need thereof, comprising: selecting a patient having a circulating tumor DNA KIT exon 17 and/or a KIT exon 18 mutation and without one or more of a KIT exon 9, 13, and 14 mutation to obtain a selected patient; administering to the selected patient 150 mg of a compound represented by:

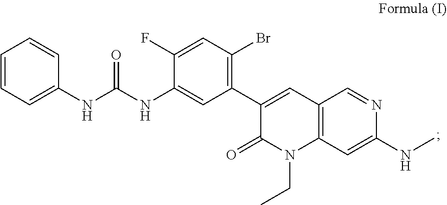 [SMILES: CCn1c(=O)c(-c2cc(NC(=O)Nc3ccccc3)c(F)cc2Br)cc2cnc(NC)cc21]

daily or twice daily; or if the patient is not a selected patient, administering to the patient one or more different kinase inhibitors.

### BRIEF DESCRIPTION OF THE DRAWINGS

FIG. 1 depicts a flow chart for the ctDNA sample evaluability conducted from randomized patient population with advanced GIST and have taken imatinib as the 1^{st} line of treatment, as described in Example 1.

FIG. 2 depicts a flow chart of detecting KIT or PDGFRA exon mutations by ctDNA test as described in Example 1.

FIG. 3 depicts PFS by IRR for GIST patients with KIT mutations at exon 11 and exons 17/18 upon treatment with ripretinib or sunitinib.

FIG. 4 depicts PFS by IRR for GIST patients with KIT mutations at exons 17/18 upon treatment with ripretinib or sunitinib.

FIG. 5 depicts a plot of a GIST interim analysis (Interim Analysis 1) of overall survivals from patients who have been administered ripretinib or sunitinib and exhibits KIT mutations at exon 11 and exon 17/18.

FIG. 6 depicts a plot of an extended GIST interim analysis (Interim Analysis 2) of overall survival from patients who have been administered ripretinib or sunitinib and exhibits KIT mutations at exon 11 and exon 17/18.

FIG. 7 depicts a plot of a GIST interim analysis (Interim Analysis 1) of overall survival from patients who have been administered ripretinib or sunitinib and exhibits KIT mutations at exon 17/18.

FIG. 8 depicts a plot of an extended GIST interim analysis (Interim Analysis 2) of overall survival from patients who have been administered ripretinib or sunitinib and exhibits KIT mutations at exon 17/18.

FIG. 9 depicts a forest plot of progression-free survival (PFS) of GIST patients by KIT mutational status from baseline ctDNA.

FIG. 10 depicts a forest plot of progression-free survival (PFS) of GIST patients by KIT exon 11 mutational subgroups from baseline ctDNA.

FIG. 11 depicts a forest plot of overall response rate (ORR) of GIST patients by KIT mutational status from baseline ctDNA.

FIG. 12 depicts a forest plot of overall response rate (ORR) of GIST patients by KIT exon 11 mutational subgroups from baseline ctDNA.

FIG. 13 depicts a forest plot of overall survival (OS) of GIST patients by KIT mutational status from baseline ctDNA during an extended interim analysis (Interim Analysis 2).

FIG. 14 depicts a forest plot of overall survival (OS) of GIST patients by KIT exon 11 mutational subgroups from baseline ctDNA during an extended interim analysis (Interim Analysis 2).

FIG. 15 shows a comparison of ORR between patients undergoing sunitinib treatment and patients undergoing ripretinib treatment in populations having KIT exon 11 and KIT exon 17/18 mutations at Data cut 1 as described in Example 1.

FIG. 16 shows a Kaplan-Meier plot of progression-free survival (PFS) of patients in a bridging study of the study of Example 1, where the patients were determined to have mutations in KIT exon 11 and exon 17 or exon 18 per the tumor tissue ITT population.

FIG. 17 shows a Kaplan-Meier analysis of PFS (A) and OS (B) for patients with ctDNA-ND versus ctDNA-D as described in Example 4.

FIG. 18 shows ORR in patients with ctDNA-ND versus ctDNA-D as described in Example 4.

FIG. 19 shows a Kaplan-Meier analysis of PFS for patients treated with ripretinib or sunitinib in the ctDNA-ND (A) and ctDNA-D (B) populations as described in Example 4.

FIG. 20 shows a forest plot of PFS by KIT mutational status as determined by local pathology report at randomization as described in Example 4.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

As used herein, “ripretinib” is a compound represented by the following structure:

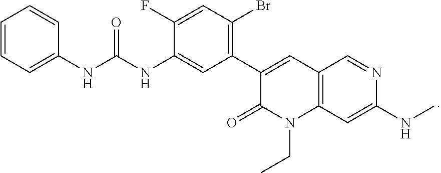 [SMILES: CCn1c(=O)c(-c2cc(NC(=O)Nc3ccccc3)c(F)cc2Br)cc2cnc(NC)cc21]

Ripretinib is also referred to herein as the compound of Formula (I).

As used herein, “sunitinib” is a compound represented by the following structure:

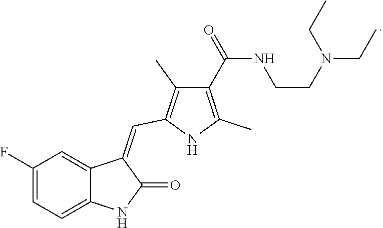 [SMILES: CCN(CC)CCNC(=O)c1c(C)[nH]c(/C=C2\C(=O)Nc3ccc(F)cc32)c1C]

As used herein, “imatinib” is a compound represented by the following structure:

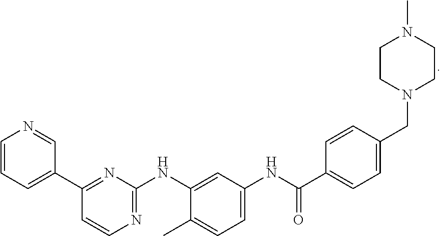 [SMILES: Cc1ccc(NC(=O)c2ccc(CN3CCN(C)CC3)cc2)cc1Nc1nccc(-c2cccnc2)n1]

As used herein, “regorafenib” is a compound represented by the following structure:

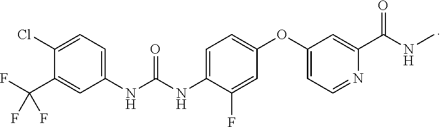 [SMILES: CNC(=O)c1cc(Oc2ccc(NC(=O)Nc3ccc(Cl)c(C(F)(F)F)c3)c(F)c2)ccn1]

“Individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

As used herein, “treating” includes any effect, e.g., lessening, reducing, modulating, or eliminating, that results in the improvement of the condition, disease, disorder and the like.

“Therapeutically effective amount” includes the amount of the subject compound that will elicit the biological or medical response of a tissue, system, animal or human that is being sought by the researcher, veterinarian, medical doctor or other clinician. A compound described herein, e.g., ripretinib is administered in therapeutically effective amounts to treat a condition described herein, e.g., gastrointestinal stromal tumors. Alternatively, a therapeutically effective amount of a compound is the quantity required to achieve a desired therapeutic and/or prophylactic effect, such as an amount which results in the prevention of or a decrease in the symptoms associated with the condition.

As used herein, “KIT” also refers to KIT proto-oncogene, c-Kit, KIT, Kit, c-kit, c-kit, or CD117.

A compound described herein, e.g., ripretinib, can be formulated as a pharmaceutical composition using a pharmaceutically acceptable carrier and administered by a variety of routes. In some embodiments, such compositions are for oral administration. In some embodiments, compositions formulated for oral administration are provided as tablets. In some embodiments, such compositions are for parenteral (by injection) administration. In some embodiments, such compositions are for transdermal administration. In some embodiments, such compositions are for topical administration. In some embodiments, such compositions are for intravenous (IV) administration. In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

As used herein, “exon 13/14” refers to i) exon 13 and exon 14 or ii) exon 13 or exon 14.

As used herein, “exon 17/18” refers to i) exon 17 and exon 18 or ii) exon 17 or exon 18.

### Methods of Treatment

In an embodiment, described herein is a method of treating a patient suffering from an advanced gastrointestinal stromal tumor, where the patient has a) a KIT exon 17 and/or a KIT exon 18 mutation and/or b) a KIT exon 11 mutation, comprising: administering to the patient 150 mg of a compound represented by:

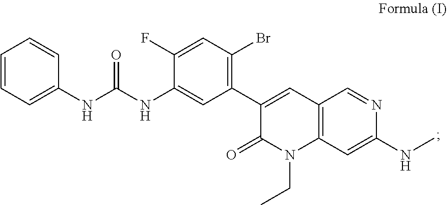 [SMILES: CCn1c(=O)c(-c2cc(NC(=O)Nc3ccccc3)c(F)cc2Br)cc2cnc(NC)cc21]

daily or twice daily, and where before the administration of the compound, the patient has progressed on or was intolerant to imatinib; and wherein upon administration of the compound the patient achieves significantly more progression free survival time as determined by mRECIST1.1, and significantly more overall survival time, as compared to a patient with a KIT exon 17 and/or a KIT exon 18 mutation and/or a KIT exon 11 mutation suffering from an advanced gastrointestinal stromal tumor who has progressed on or was intolerant to imatinib and was then administered 50 mg sunitinib once daily.

In some embodiments the patient has a) the KIT exon 17 and/or the KIT exon 18 mutation and/or b) the KIT exon 11 mutation in circulating tumor DNA. In some embodiments, the patient being administered the compound does not have a KIT exon 9, or does not have a KIT exon 13 or 14 mutation in circulating tumor DNA. In some embodiments, the patient being administered the compound does not have a KIT exon 9, and does not have a KIT exon 13 or 14 mutation in circulating tumor DNA. In some embodiments, the patient being administered the compound has a progression free survival of at least about 14 months as compared to a progression free survival of about 1.5 months for the patient administered sunitinib. In some embodiments, the method comprises administering 150 mg of the compound once or twice daily for at least 40 days to the patient. In some embodiments, the method comprises administering 150 mg of the compound to the patient once daily.

In another embodiment, described herein is a method of treating a patient suffering from an advanced gastrointestinal stromal tumor who has a KIT exon 17 and/or a KIT exon 18 mutation, does not have KIT exon 13 and/or 14 mutation, and does not have a KIT exon 9 mutation, and where the patient has progressed on or was intolerant to imatinib, comprising administering to the patient 150 mg of a compound represented by:

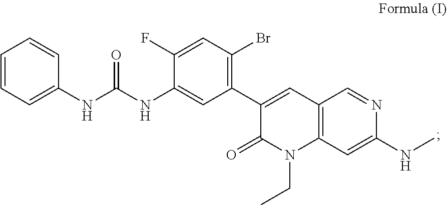 [SMILES: CCn1c(=O)c(-c2cc(NC(=O)Nc3ccccc3)c(F)cc2Br)cc2cnc(NC)cc21]

daily or twice daily.

In some embodiments, the patient has the KIT exon 17 and/or a KIT exon 18 mutation, does not have the KIT exon 13 and/or 14 mutation, and does not have the KIT exon 9 mutation, in circulating tumor DNA. In some embodiments, the patient's circulating tumor DNA also has a KIT exon 11 mutation. In some embodiments, the method comprises administering to the patient 150 mg of the compound daily.

In another embodiment, described herein is a method of treating a patient suffering from an advanced gastrointestinal stromal tumor where the patient has progressed on or was intolerant to imatinib, and the patient's circulating tumor DNA has a KIT exon 17 and/or a KIT exon 18 mutation and does not have a KIT exon 9 mutation, comprising administering to the patient 150 mg of a compound represented by:

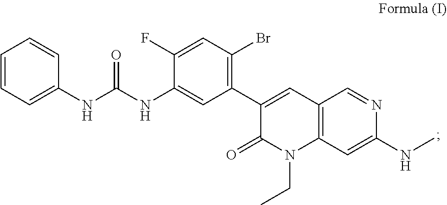 [SMILES: CCn1c(=O)c(-c2cc(NC(=O)Nc3ccccc3)c(F)cc2Br)cc2cnc(NC)cc21]

daily or twice daily.

In some embodiments, the method comprises administering to the patient 150 mg of the compound daily.

In another embodiment, described herein is a method of treating an advanced gastrointestinal stromal tumor where the patient has progressed on or was intolerant to imatinib in a patient in need thereof, comprising: selecting a patient having a circulating tumor DNA KIT exon 17 and/or a KIT exon 18 mutation and without one or more of a KIT exon 9, 13, and 14 mutation to obtain a selected patient; administering to the selected patient 150 mg of a compound represented by:

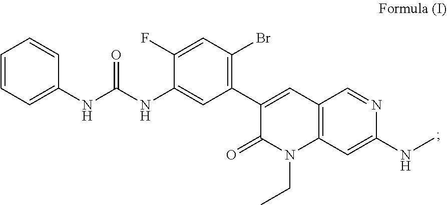 [SMILES: CCn1c(=O)c(-c2cc(NC(=O)Nc3ccccc3)c(F)cc2Br)cc2cnc(NC)cc21]

daily or twice daily; or if the patient is not a selected patient, administering to the patient one or more different kinase inhibitors.

In some embodiments, the one or more different kinase inhibitors is selected from the group consisting of sunitinib, regorafenib, lapatinib, gefitinib, erlotinib, vatalanib, and crenolanib. In some embodiments, the one or more different kinase inhibitors is selected from the group consisting of sunitinib and regorafenib. In some embodiments, the one or more different kinase inhibitors is selected from the group consisting of sunitinib malate and regorafenib.

In some embodiments, if the patient is not a selected patient, the one or more different kinase inhibitors includes sunitinib. In some embodiments, if the patient is not a selected patient, comprising administering to the patient 50 mg of sunitinib once daily. In some embodiments, if the patient is not a selected patient and has further progressed at least after the sunitinib administration, further comprising administering to patient 150 mg of the compound daily or twice daily. In some embodiments, the method comprises administering to the selected patient 150 mg of the compound once daily.

In some embodiments, the therapeutic efficacy of ripretinib is determined by the progression-free survival of the patient after independent radiologic review using Response Evaluation Criteria in Solid Tumors (RECIST). In some embodiments, the therapeutic efficacy of ripretinib is determined by the progression-free survival of the patient after independent radiologic review using modified Response Evaluation Criteria in Solid Tumors (mRECIST). In some embodiments, the therapeutic efficacy of ripretinib is determined by the Objective Response Rate (ORR), Time to Tumor Progression (TTP) or Overall Survival (OS) of the patient after independent radiologic review using mRECIST. In some embodiments, the therapeutic efficacy of ripretinib is determined by the progression-free survival of the patient based on investigator assessment. In some embodiments, the therapeutic efficacy of ripretinib is determined by the quality of life of the patient in accordance with European Organisation for Research and Treatment of Cancer Quality of Life Questionnaire for Cancer 30-item (EORTC-QLQ-C30) and the EuroQol 5-Dimension 5-Level (EQ-5D-5L) questionnaires. In some embodiments, the therapeutic efficacy of ripretinib is determined by the disease control rate of the patient. In some embodiments, the therapeutic efficacy of ripretinib is determined by the duration of response of the patient. In some embodiments, the therapeutic efficacy of ripretinib is evaluated within one month of administering the ripretinib (e.g., at 1, 15, or 29 days). In some embodiments, the therapeutic efficacy of ripretinib is evaluated within three months of administering the ripretinib (e.g., at 43, 71, or 85 days). In some embodiments, the therapeutic efficacy of ripretinib is evaluated after three months of administering the ripretinib.

After at least one month, two months, e.g., 42 days or more of treatment with ripretinib, the patient may have a progression-free survival as measured using mRECIST v1.1. As another example, the patient may have a least a 5- or 6-month progression-free survival as compared to placebo after at least 4 weeks of daily administration of ripretinib, and/or for example, after 4 weeks of daily administration of ripretinib, significantly reduced the risk of disease progression or death by 85%.

In some embodiments, the patient has at least one measurable tumor lesion according to modified RECIST Version 1.1 within 21 days prior to the first dose of ripretinib. In some embodiments, the patient has a non-nodal tumor lesion of greater than or equal to 1.0 cm in the long axis or greater than or equal to double the slide thickness in the long axis, within 21 days prior to the first dose of ripretinib.

### Dose Modifications

Dose modifications may be made in the methods of administering ripretinib described herein as a result of adverse events experienced by the patient. In some embodiments, the dose modification is a dose interruption. In some embodiments, the dose modification is a permanent discontinuation in dosing. In some embodiments, the dose modification is a dose reduction. In some embodiments, the dose of ripretinib administered to the patient is reduced from 150 mg once daily, e.g., three tablets each comprising 50 mg of ripretinib, to 100 mg once daily, e.g., two tablets each comprising 50 mg of ripretinib. In some embodiments, the dose of ripretinib administered to the patient is reduced from 150 mg once daily, e.g., three tablets each comprising 50 mg of ripretinib, to 50 mg once daily, e.g., one tablet comprising 50 mg of ripretinib. In some embodiments, the adverse reaction is selected from the group consisting of a hand-foot skin reaction (e.g., palmar-plantar erythrodysesthesia syndrome), hypertension, arthralgia, and myalgia.

In some embodiments, the adverse event is graded in accordance with the National Cancer Institute Common Terminology Criteria for Adverse Events, version 5.0 (e.g., baseline, Grade 1, Grade 2, Grade 3, or Grade 4). In some embodiments, the dose modification is a dose interruption (e.g., a dose interruption of at least 7 days) as a result of a Grade 2 adverse event. In some embodiments, dosing resumes at the same dose level before the dose interruption if the adverse event is lowered to Grade 1 or baseline within a first time period (e.g., within 7 days). In some embodiments, dosing resumes at a reduced dose level before the dose interruption if the adverse event is lowered to Grade 1 or baseline after a first time period (e.g., after 7 days). In some embodiments, the reduced dose level is re-escalated to the dose level prior to the dose interruption if the adverse event is lowered to Grade 1 or baseline after a first time period but is maintained as a Grade 1 or baseline adverse event after a second time period (e.g., after 28 days). In some embodiments, the dose modification is a dose interruption (e.g., a dose interruption of at least 7 days up to a maximum of 28 days) as a result of a Grade 3 adverse event. In some embodiments, dosing is continued at a reduced level after the dose interruption. In some embodiments, the dose modification is a permanent discontinuation in dosing as a result of a Grade 4 adverse event (e.g., Grade 4 hypertension).

A patient can be administered an additional treatment in response to an adverse event or to prevent an adverse event from occurring. In some embodiments, a patient suffering from an adverse dermatologic reaction, e.g., a hand-foot skin reaction, e.g., palmar-plantar erythrodysesthesia syndrome, is administered a topical composition (e.g., an emollient) to treat the adverse dermatologic reaction. In some embodiments, the patient is administered the topical composition (e.g., an emollient) based on the severity of the adverse dermatologic reaction, e.g., a Grade 2, Grade 3 adverse dermatologic reaction, e.g., a Grade 1, Grade 2, or Grade 3 hand-foot skin reaction, e.g., a Grade 1, Grade 2 or Grade 3 palmar-plantar erythrodysesthesia syndrome. In some embodiments, the topical composition (e.g., an emollient) is administered to the patient during a dose interruption of ripretinib. In some embodiments, the topical composition (e.g., an emollient) is administered to the patient contemporaneously with a dose of ripretinib, e.g., a reduced dose of ripretinib.

A patient can also be administered an additional treatment prior to, or during administration of ripretinib in accordance with the methods described herein to prevent or ameliorate an adverse event. In some embodiments, the patient is administered a topical composition (e.g., an emollient) before and/or during ripretinib administration to prevent or ameliorate the onset of an adverse dermatologic reaction, e.g., a hand-foot skin reaction, e.g., palmar-plantar erythrodysesthesia syndrome.

### EXAMPLES

### Example 1. Mutational Heterogeneity of Imatinib Resistance and Efficacy of Ripretinib Versus Sunitinib in Patients With Gastrointestinal Stromal Tumor: ctDNA Analysis From a Multicenter, Global, Randomized, Open-Label, Phase 3 Study

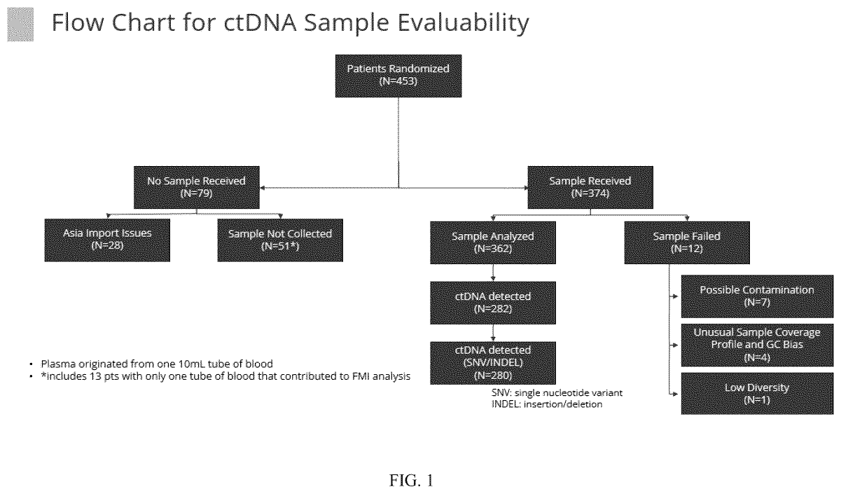

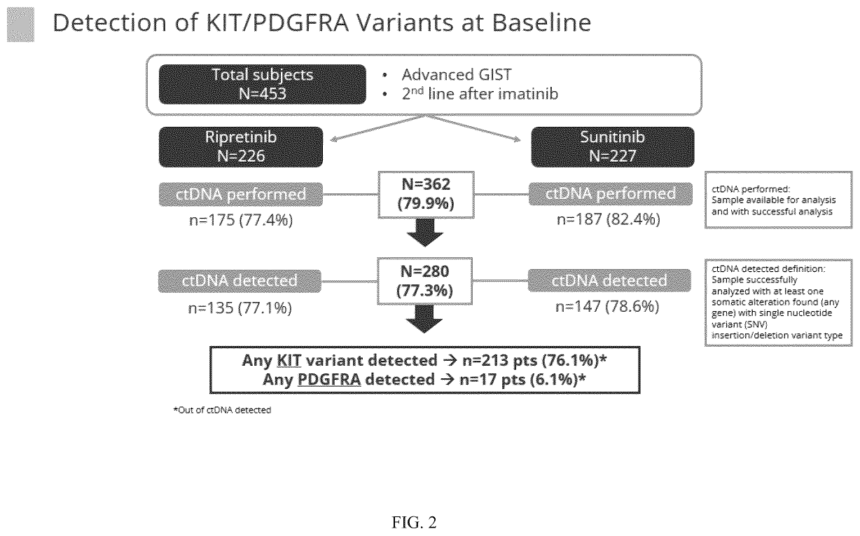

This study is a multicenter, global, randomized, open-label, phase 3 study which enrolled adult patients with advanced gastrointestinal stromal tumor (GIST) who progressed on or had intolerance to imatinib. Randomization was 1:1 to ripretinib 150 mg once daily (QD) or sunitinib 50 mg once daily (QD) (4 weeks on/2 weeks off). Baseline peripheral whole blood was analyzed by Guardant360, a 74-gene circulating tumor DNA (ctDNA) next-generation sequencing (NGS)-based assay. The flow chart for the ctDNA sample evaluability is shown in FIG. 1. A scheme for conducting ctDNA tests for detecting exon mutations at baseline is shown in FIG. 2.

Of the 453 patients in the overall intent-to-treat population (ITT), baseline ctDNA analysis was performed in 362 patients for whom evaluable samples were available. ctDNA was detected in 280 samples and KIT mutations were detected in 213 patients. Primary mutations in KIT were detected in exon 11 in 157 patients and in exon 9 in 36 patients. Common resistance mutations in KIT were in exons 17/18 in 89 patients and exons 13/14 in 81 patients. In patients with a KIT exon 11 primary mutation, 52 patients had mutations in exon 17 or 18 only, 41 had mutations in exon 13 or 14 only, and 22 patients had mutations in both exon 13 or 14 and exon 17 or 18. Patients with KIT exon 11 only primary mutation and exon 17 or 18 secondary mutations only had superior progression-free survival (PFS), objective response rate (ORR), and overall survival (OS) with QINLOCK versus sunitinib (Table 1). Efficacy in patients with detectable ctDNA in KIT exon 11 and the ITT populations was consistent with the primary analysis based on tumor data used for randomization. The subgroup safety profiles were consistent with the primary analysis.

TABLE 1
Summary of Efficacy Results of ctDNA Analysis for Patients
with Mutations in KIT Exon 11 and 17 or 18 Only

| | | | Hazard |
|---|---|---|---|
| | | | Ratio/Response |
| | Ripretinib | Sunitinib | Difference |
| | (n = 27) | (n = 25) | (95% CI) |
| Median Progression- | 14.2 months | 1.5 months | 0.22 (0.11, 0.44) |
| Free Survival (mPFS)⁽¹⁾ | | | |
| ORR⁽¹⁾ | 44.4% | 0% | 44.4% (23.0%, 62.7%) |
| OS⁽²⁾ | Not Estimable | 17.5 months | 0.34 (0.15, 0.76) |

Notes:
⁽¹⁾Data cut 1;
⁽²⁾Data cut 2.

### Topline Results

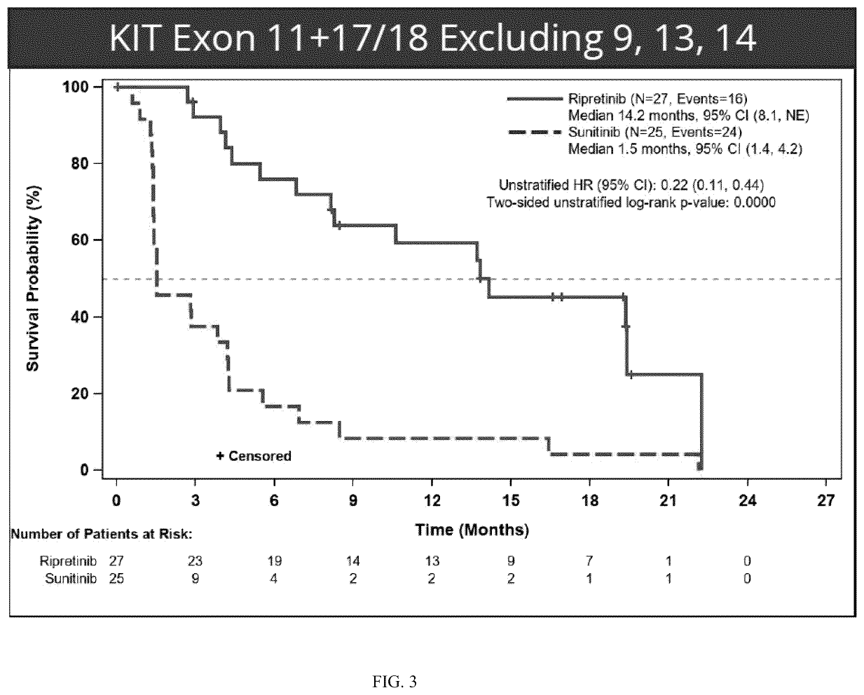

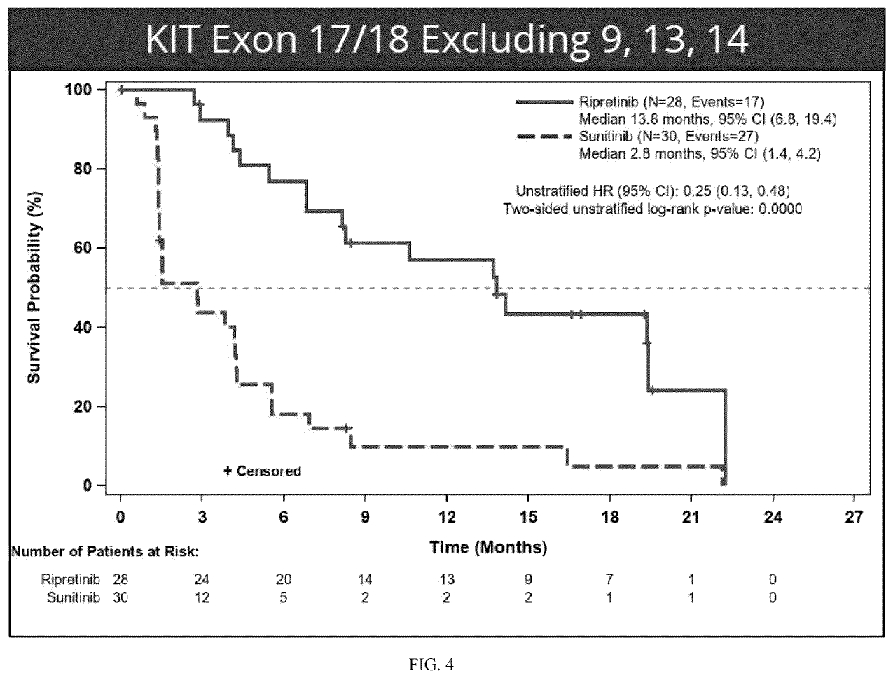

PFS plots by independent radiologic review (IRR) for patients with GIST having KIT mutations at exon 11 and exon 17/18, excluding mutations in KIT exons 9, 13, and/or 14, is shown in FIG. 3, in which patients administered ripretinib demonstrated a median PFS of 14.2 months, whereas patients administered sunitinib demonstrated a median PFS of 1.5 months. PFS plots by IRR for patients with GIST having for patients with KIT mutations at exon 17/18, excluding mutations in KIT exons 9, 13, and/or 14, is shown in FIG. 4, in which patients administered ripretinib demonstrated a median PFS of 13.8 months, whereas patients administered sunitinib demonstrated a median PFS of 2.8 months.

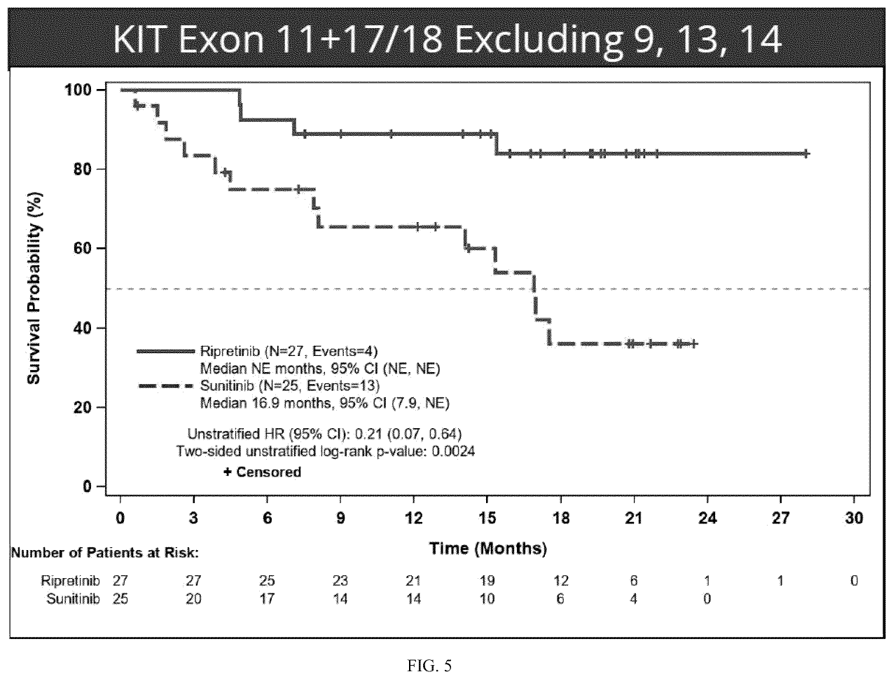

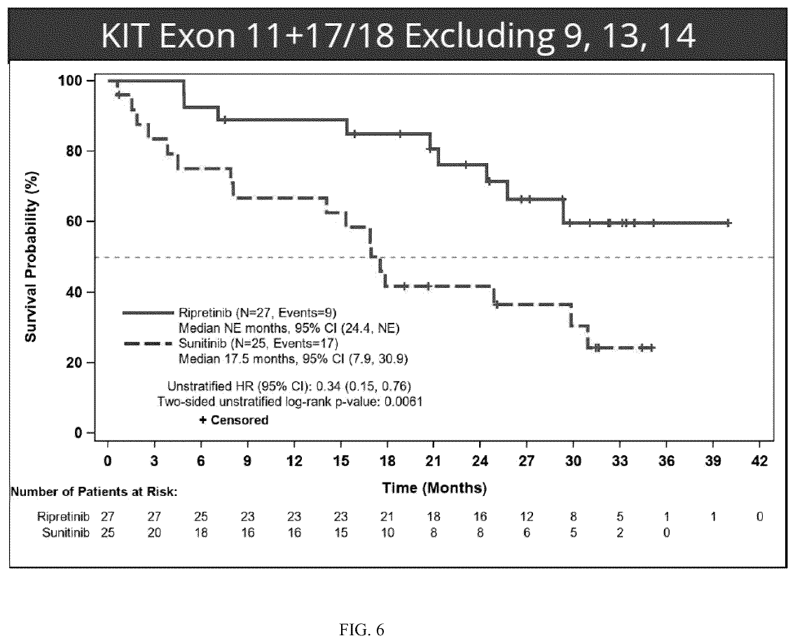

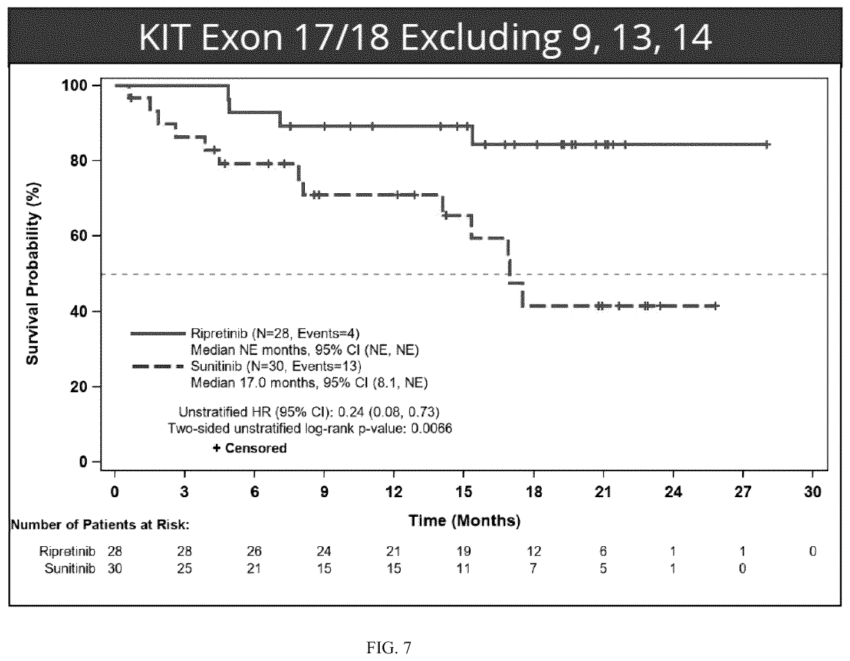

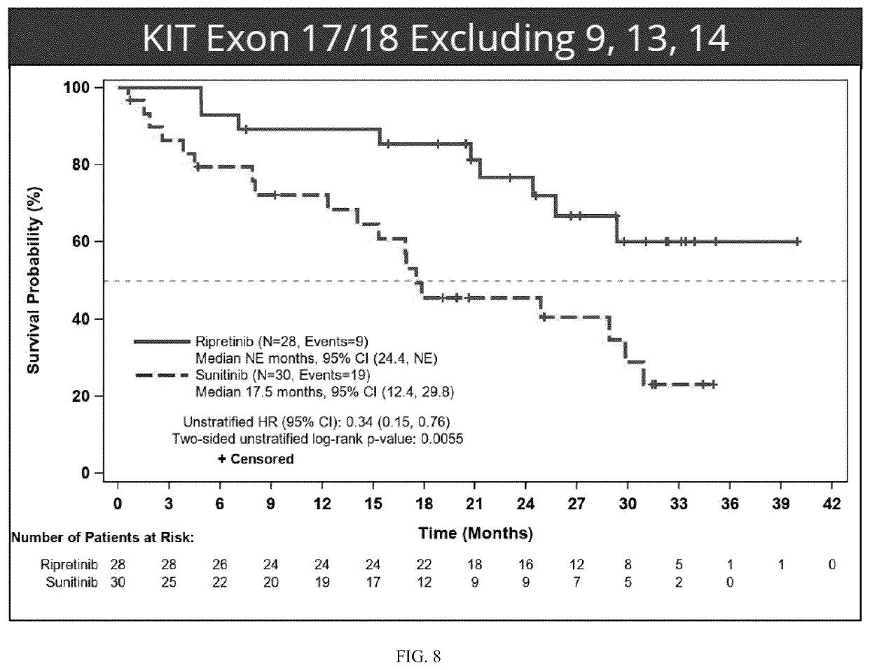

FIG. 5 illustrates an interim analysis of overall survival (OS) of patients with GIST having mutations at KIT exon 11 and exon 17/18, excluding mutations in KIT exons 9, 13, and/or 14, in which patients administered ripretinib demonstrated a median OS that was not estimable (NE), whereas patients administered sunitinib demonstrated a median OS of 16.9 months. FIG. 6 illustrates an extended interim analysis of overall survival (OS) of patients with GIST having mutations at KIT exon 11 and exon 17/18, excluding mutations in KIT exons 9, 13, and/or 14, in which patients administered ripretinib demonstrated a median OS that was not estimable (NE), whereas patients administered sunitinib demonstrated a median OS of 17.5 months. FIG. 7 illustrates an interim analysis of overall survival (OS) of patients with GIST having mutations at KIT exon 17/18, excluding mutations in KIT exons 9, 13, and/or 14, in which patients administered ripretinib demonstrated a median OS that was not evaluable (NE), whereas patients administered sunitinib demonstrated a median OS of 17.0 months. FIG. 8 illustrates an extended interim analysis of overall survival (OS) of patients with GIST having mutations at KIT exon 17/18, excluding mutations in KIT exons 9, 13, and/or 14, in which patients administered ripretinib demonstrated a median OS that was not evaluable (NE), whereas patients administered sunitinib demonstrated a median OS of 17.5 months.

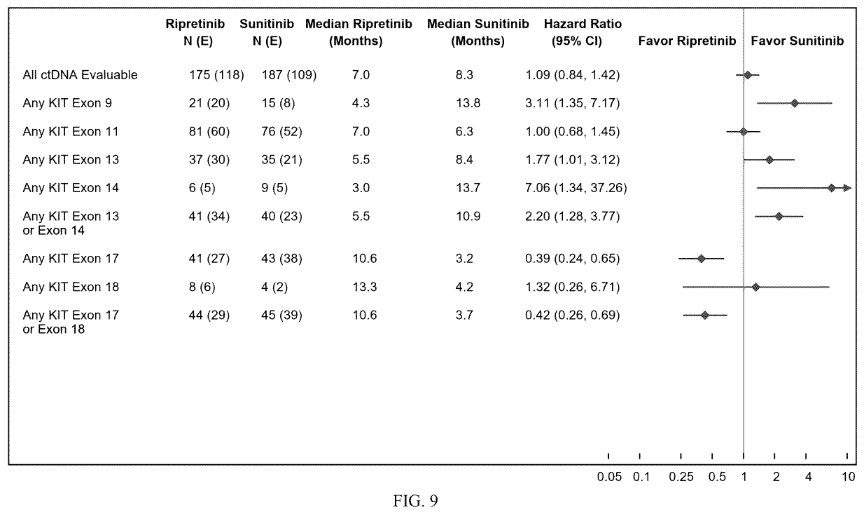

FIG. 9 depicts a forest plot of progression-free survival (PFS) of GIST patients by KIT mutational status from baseline ctDNA. The plots show that mutations in KIT exons 17 and 18 favor ripretinib treatment, while mutations in KIT exons 9, 13, and 14 favor sunitinib.

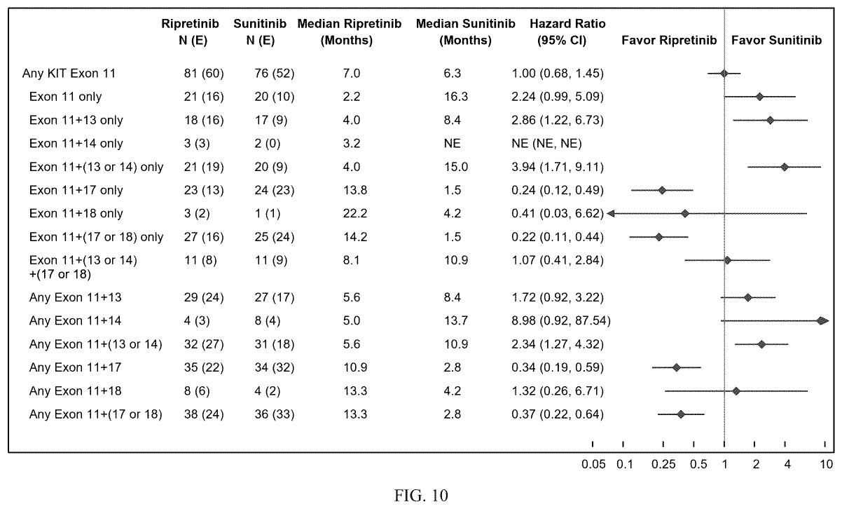

FIG. 10 depicts a forest plot of progression-free survival (PFS) of GIST patients by KIT exon 11 mutational subgroups from baseline ctDNA. The plots show that, for example, populations with mutations in KIT exon 11 and KIT exon 17 only, KIT exon 11 and KIT exon 18 only, any case where there is a mutation in KIT exons 11 and 17, and any case where there is a mutation in exons 11 and exon 17 or 18 favor ripretinib treatment. However, the plots favor sunitinib for exon 11 mutations in combination with exon 13 or exon 14 mutations.

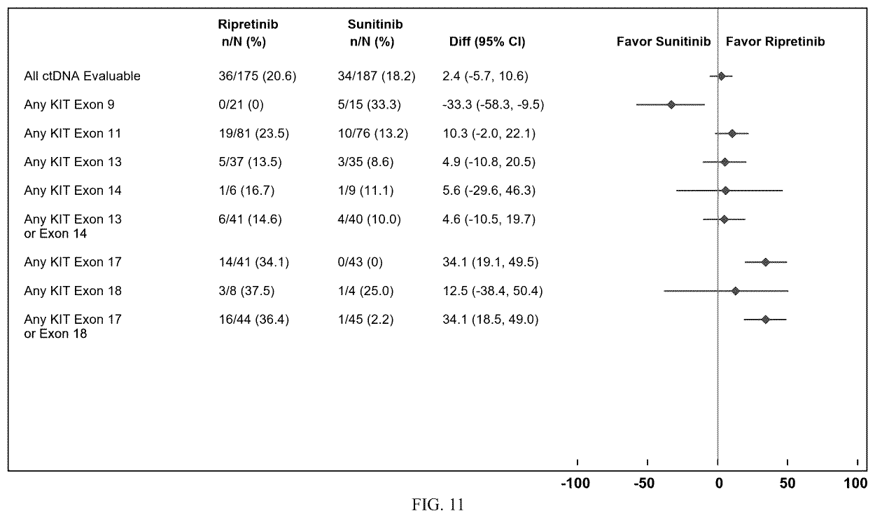

FIG. 11 depicts a forest plot of overall response rate (ORR) of GIST patients by KIT mutational status from baseline ctDNA. For example, the plot favors ripretinib treatment for populations with KIT exons 11, 17 or 18. However, the plot favors sunitinib treatment for, e.g., KIT exon 9 mutations.

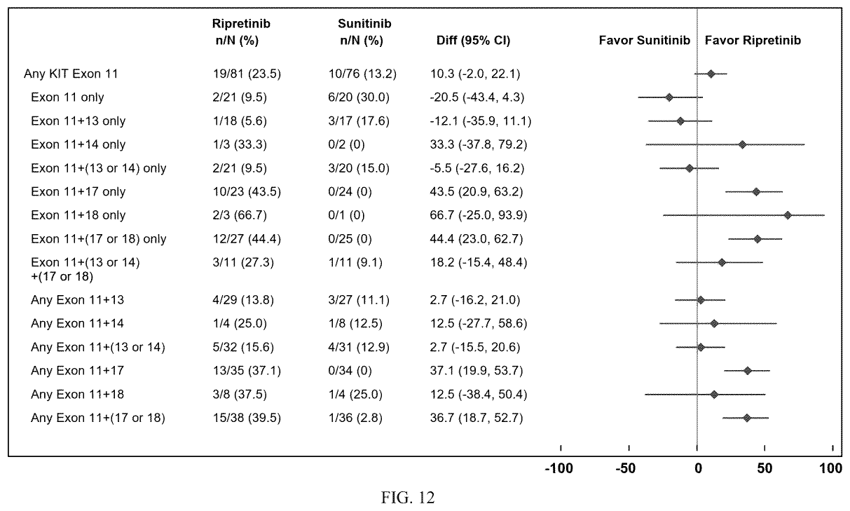

FIG. 12 depicts a forest plot of overall response rate (ORR) of GIST patients by KIT exon 11 mutational subgroups from baseline ctDNA. For example, the plot favors ripretinib treatment for populations with KIT exon 11 mutations in combination with KIT exon 17 or 18 mutations, while the plot favors sunitinib treatment for, e.g., KIT exon 11 mutations in combination with KIT exon 13 only.

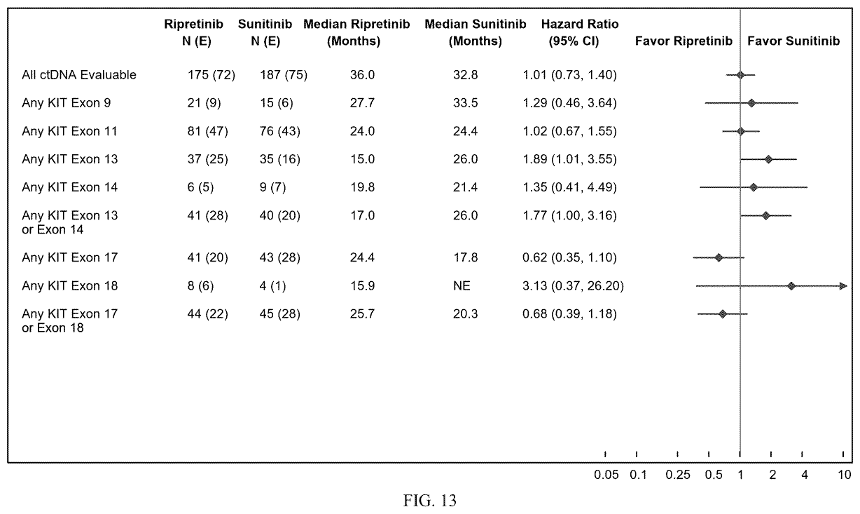

FIG. 13 depicts a forest plot of overall survival (OS) of GIST patients by KIT mutational status from baseline ctDNA during an extended interim analysis (Interim Analysis 2). For example, the plot favors ripretinib treatment for populations with KIT exon 17 and 18 mutations, but the plot favors sunitinib treatment for, e.g., KIT exon 13 and 14 mutations.

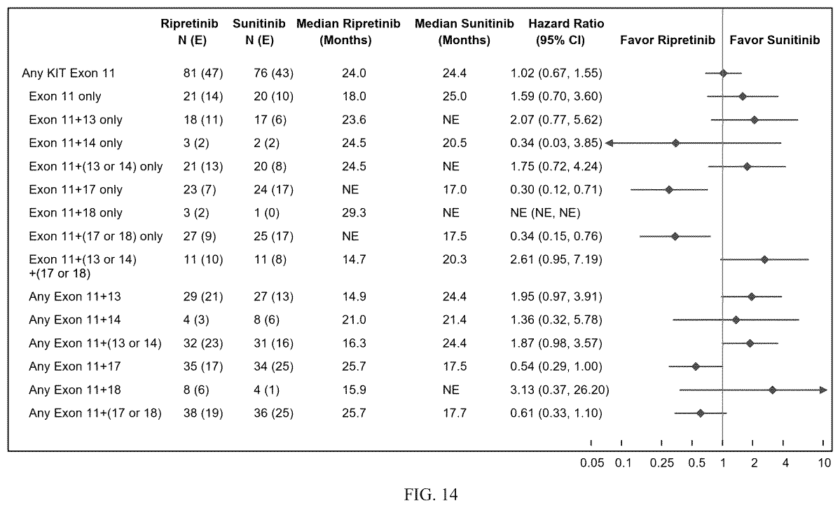

FIG. 14 depicts a forest plot of overall survival (OS) of GIST patients by KIT exon 11 mutational subgroups from baseline ctDNA during an extended interim analysis (Interim Analysis 2). For example, the plot favors ripretinib treatment for populations with KIT exon 11 and 17 or KIT exon 11 and 18 mutations. In contrast, the plot favors sunitinib treatment for populations having, for example, KIT exon 11 and 13 mutations or KIT exon 11 and 14 mutations.

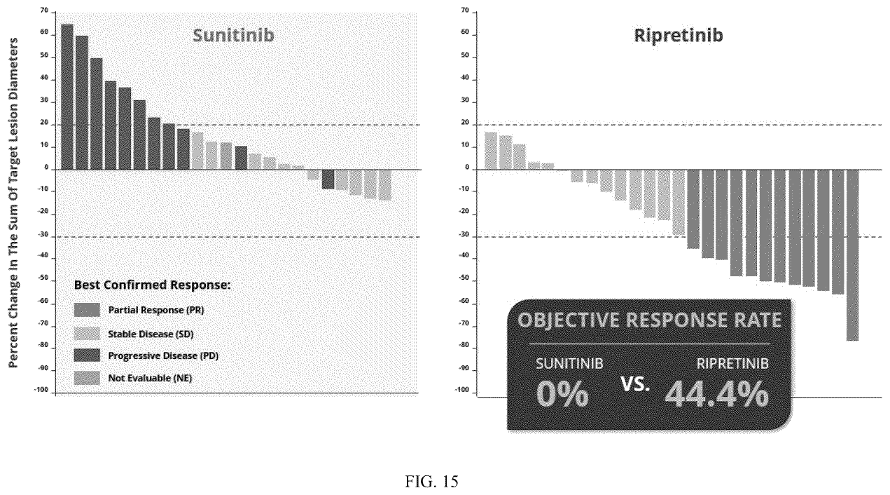

In addition, FIG. 15 shows a comparison of ORR between patients undergoing sunitinib treatment and patients undergoing ripretinib treatment in populations having KIT exon 11 and KIT exon 17/18 mutations at Data cut 1 as described above. The comparison shows an ORR of 44.4% for ripretinib, whereas the ORR is 0% for sunitinib.

### Example 2. A Phase 3, Randomized, Multicenter, Open-Label Study of Ripretinib 150 mg QD Versus Sunitinib in Second-Line Advanced GIST Patients With KIT Exons 11+17/18 Mutation, After Treatment With Imatinib

This is a Phase 3 clinical trial that constitutes 54 patients with advanced gastrointestinal stromal tumor (GIST), who have been previously treated with imatinib. The resulting patient population will undergo 2:1 randomization into the ripretinib treatment group (N=36) or sunitinib (N=18) treatment, Patients randomized to sunitinib arm may crossover to ripretinib arm after progressive disease. Inclusion criteria include: male or female 18 years of age or older; histologic diagnosis of GIST with co-occurring KIT exon 11+17/18 mutations confirmed by central laboratory ctDNA analysis at pre-screening; advanced GIST and radiologic progression on imatinib treatment, which was discontinued 10 days or more prior to receiving the first dose of study drug; patient must have at least one measurable lesion according to mRECIST v1.1 within 21 days prior to first dose of study drug; and Eastern Cooperative Oncology Group performance status (ECOG PS) of greater than or equal to 2. Exclusion criteria include: co-occurring KIT exon 11+17 and/or 18 mutations that cannot be confirmed by central laboratory ctDNA analysis; history of KIT exon 9 mutation or detection of KIT exon 9, 13, or 14 mutations by central laboratory ctDNA analysis; treatment with any other line of therapy in addition to imatinib for advanced GIST (imatinib-containing combination therapy in the first-line setting is not allowed); any prior or concurrent malignancy whose treatment may interfere with safety or efficacy assessment of this study; and known active metastasis of the central nervous system.

Patients who are administered ripretinib will receive 150 mg QD, and those who are administered sunitinib will receive 50 mg QD by receiving 4 weeks of sunitinib treatment, and 2 weeks of no sunitinib treatment. The 150 mg QD of ripretinib (3×50 mg tablets) will be dosed continuously in repeated 42-day cycles, while the 50 mg QD of sunitinib (4×12.5 mg capsules) will be dosed in 42-day cycles in which sunitinib will be given continuously for 4 weeks with a 2-week break. Participants will visit the study site as follows: Days 1, 15, and 29 of Cycle 1; Days 1 and 29 of Cycle 2; and Day 1 only of Cycle 3 and all other subsequent Cycles. Both treatments will proceed until disease progression, unacceptable toxicity, or withdrawal of patients' study consent. The primary endpoint will be progression-free survival (PFS) by independent radiologic review (IRR) using mRECIST. Key secondary endpoints include objective response rate (ORR) by IRR using mRECIST and overall survival (OS).

Other secondary endpoints include summary measures from the EORTC-QLQ-C30, NCI-PRO-CTCAE items (questions number 15 “constipation”, 16 “diarrhea”, 30 “hand-foot syndrome”, and 53a and b “fatigue”), and the EQ-5D-5L; time-to-progression (TTP) based on IRR per mRECIST, which is defined as the time from randomization until documented progressive disease (PD) based on IRR per mRECIST; disease control rate (DCR) at 6, 12, 18, and 24 weeks based on IRR per mRECIST, which is defined as the proportion of participants who achieve complete response (CR), partial response (PR), or stable disease (SD) at the specified time point based on IRR per mRECIST; PFS based on the Investigator per mRECIST, which is defined as the time from randomization until documented PD based on Investigator's assessment per mRECIST or death due to any cause, whichever occurs first; duration of response (DOR) for participants who achieve confirmed CR or PR, defined as the time interval from the time that the measurement criteria are first met for confirmed CR or confirmed PR (whichever is first recorded) until the first date the PD is objectively documented or death, whichever occurs first; and TTR is defined as the time from date of randomization until the first assessment of confirmed CR or PR per mRECIST.

Safety endpoints include frequency of treatment-emergent adverse events (TEAEs); frequency of serious adverse events (SAEs); frequency of TEAEs leading to dose reduction, interruption, or discontinuation of study drugs; and changes from baseline in ECOG PS, vital signs, ECGs, dermatologic examinations, and clinical laboratory parameters.

Exploratory endpoints include pharmacokinetics (PK) of ripretinib including Cₘₐₓ, Tₘₐₓ, T_{1/2}, and AUC; PFS2, which is defined as the time from randomization until PD on next-line treatment as determined by the Investigator, or death due to any cause, whichever occurs first; second PFS, which is defined as the date of the first dose of next-line treatment until PD based on Investigator assessment or death due to any case, whichever occurs first; participant interview to complete the healthcare utilization questionnaire (HCUQ); baseline levels and change in select plasma biomarkers; and association of genetic variations in the population with differences in PK, pharmacodynamics, efficacy, tolerability, and/or safety.

### Ripretinib Interruption and Modification Due to Toxicity

Ripretinib may be interrupted or reduced from a first dose reduction of 150 mg once daily to 100 mg once daily or a second dose reduction to 50 mg once daily. These dose interruptions or reductions are made at the discretion of the Investigator due to adverse events and according to the criteria for interruption or reduction of ripretinib in Table 2 (dermatologic toxicities, arthralgia, and myalgia), Table 3 (left ventricular systolic dysfunction), Table 4 (hypertension), and Table 5 (treatment-related adverse events other than dermatologic toxicities, arthralgia/myalgia, left ventricular systolic dysfunction, and hypertension). The severity of an adverse event that does not appear in the National Cancer Institute Common Terminology Criteria for Adverse Events (NCI CTCAE), v5.0 scale must be assessed according to the criteria in Table 6.

If ripretinib is interrupted and then restarted, the participant should remain on their original cycle schedule. If any participant requires a ripretinib dose lower than 50 mg once daily or if a participant has had their dose reduced and has progressive disease as assessed by independent radiologic review (IRR), the participant must be discontinued from ripretinib, and the End of Treatment (EOT) Visit. Safety Follow-up, and Survival Follow-up must be conducted.

TABLE 2
Ripretinib Dose Modifications for Dermatologic Toxicities, Arthralgia, and Myalgia

| Toxicity Grade | Dose Modification Guide |
|---|---|
| Grade 1 | Institute support measures and continue ripretinib at the current dose. |
| Grade 2 | Institute support measures and continue ripretinib at the current dose. |
| | If no improvement within 7 days, then interrupt ripretinib. |
| | If the event returns to Grade 1 or baseline within 7 days, resume |
| | ripretinib at the same dose level. |
| | If the event returns to Grade 1 or baseline after 7 days, resume |
| | ripretinib at a reduced dose level. |
| | If this is a recurrence, after event returns to Grade 1 or baseline, |
| | resume ripretinib at a reduced dose level regardless of time to |
| | improvement. |
| | If after a dose reduction the event is maintained at Grade 1 or baseline |
| | for at least 1 cycle (42 days) of dosing, consider re-escalating ripretinib |
| | by 1 dose level. |
| Grade 3 | Institute support measures. |
| | Interrupt ripretinib for at least 7 days or until the event returns to |
| | Grade 1 or baseline (maximum 28 days). |
| | Resume ripretinib at a reduced dose level. |
| | If after a dose reduction the event is maintained at Grade 1 or baseline |
| | for at least 1 cycle (42 days) of dosing, consider re-escalating ripretinib |
| | by 1 dose level. |
| Grade 4 | Discontinue ripretinib unless the event is not considered life threatening. |
| | Participants may continue ripretinib with non-life-threatening events upon |
| | discussion with the Sponsor. |

TABLE 3
Ripretinib Dose Modifications for Left
Ventricular Systolic Dysfunction

| Toxicity Grade | Management Guideline |
|---|---|
| Any Grade 3 or 4 | Permanently discontinue ripretinib |

TABLE 4
Ripretinib Dose Modifications and Management of Hypertension

| Toxicity Grade | Management Guideline^{a} |
|---|---|
| Grade 1 | Continue BP monitoring |
| Prehypertension (systolic BP 120- | Continue ripretinib at the current dose level |
| 139 mmHg or diastolic BP 80-89 | |
| mmHg) | |
| Grade 2 | Treat BP to achieve diastolic BP ≤90 mmHg |
| Systolic BP 140-159 mmHg or | and or systolic BP ≤140 mmHg |
| diastolic BP 90-99 mmHg | If BP was previously within normal limits, |
| Or | start antihypertensive monotherapy |
| Symptomatic increase | If participant is already on antihypertensive |
| by >20 mmHg (diastolic) or | medication, titrate dose up |
| to >140/90 mmHg if previously | Continue the ripretinib at the current dose level |
| within normal limits | Hold ripretinib if symptomatic increase by 20 |
| | mmHg (diastolic BP) until symptoms resolve and |
| | diastolic BP ≤90 mmHg |
| | On resuming ripretinib, continue at the same dose |
| | level |
| Grade 3 | Treat to achieve diastolic BP ≤90 mmHg and or |
| Systolic BP ≥160 mmHg or | systolic BP ≤140 mmHg |
| diastolic BP ≥100 mmHg) | Start antihypertensive |
| Or | medication and/or |
| More than 1 drug or more intensive | Increase current antihypertensive |
| therapy than previously indicated | medication and/or |
| | Add additional antihypertensive medication |
| | If symptomatic, hold ripretinib until diastolic |
| | BP ≤90 mmHg and/or systolic BP ≤140 mmHg, and |
| | symptoms resolve |
| | On resuming ripretinib, continue at the same dose |
| | level |
| | If BP is not controlled with addition of a new or |
| | more intensive therapy, reduce ripretinib by 1 |
| | dose level |
| | If Grade 3 hypertension recurs despite ripretinib |
| | dose reduction and antihypertensive therapy, reduce |
| | ripretinib by 1 additional dose level |
| Grade 4 | Treat BP per institutional guidelines |
| Life-threatening consequences | Discontinue ripretinib |
| (e.g., malignant hypertension, | |
| transient or permanent neurologic | |
| deficit, and hypertensive crisis) | |

BP: blood pressure
^{a}If BP remains controlled for at least 28 days, ripretinib dose re-escalation is permitted at the Investigator's discretion.

TABLE 5
Ripretinib Dose Modifications for Treatment-related Adverse
Events Other than Dermatologic Toxicities, Arthralgia/Myalgia,
Left Ventricular Systolic Dysfunction, and Hypertension

| Toxicity Grade | Management Guideline^{a} |
|---|---|
| Any Grade 3 or 4 | Interrupt ripretinib dosing until event has |
| | resolved to Grade 1 or baseline |
| | Once the adverse event returns to Grade 1 or |
| | baseline, reduce ripretinib by 1 dose level |
| | If a participant tolerates the reduced dose without |
| | recurrence of the event for at least 28 days, consider re- |
| | escalating the dose of ripretinib to the prior dose level |
| Asymptomatic/Not clinically | Closely monitor for clinical symptoms and continue |
| significant Grade 3 or 4 | ripretinib at the current dose level. Repeat laboratory |
| laboratory AEs (including CPK | tests within 10 days |
| and lipase) that persist ≤10 days | |
| Asymptomatic/Not clinically | Closely monitor for clinical symptoms; for Grade 4 |
| significant Grade 3 or 4 | events, interrupt ripretinib until the event returns to |
| laboratory AEs (including CPK | Grade 3 |
| and lipase) that persist >10 days | Asymptomatic Grade 3 or Grade 4 elevations of |
| | plasma enzyme: lipase or CPK do not require dose |
| | interruption |
| | Once the adverse event returns to Grade 3, resume |
| | ripretinib at the current dose level or consider |
| | reducing by 1 dose level per Investigator discretion |
| | after discussion with the Sponsor |
| Clinically Significant Grade 3 | Interrupt ripretinib. If the Investigator assesses that |
| or 4 laboratory AEs (including | restarting ripretinib is in the participant's best interest, |
| CPK and lipase) | the Sponsor must be contacted to discuss the case and |
| | determine if restarting should be allowed |

CPK: creatine phosphokinase.
^{a}The rules for dose modifications for laboratory adverse events will be based on local laboratory results.

TABLE 6
Severity Grading Scale

| Classification | Definition |
|---|---|
| Grade 1 (Mild) | Asymptomatic or mild symptoms; clinical or diagnostic observations |
| | only; intervention not indicated. |
| Grade 2 (Moderate) | Minimal, local, or noninvasive intervention indicated; limiting |
| | age-appropriate instrumental ADL. |
| Grade 3 (Severe) | Severe or medically significant but not immediately life-threatening; |
| | hospitalization or prolongation of hospitalization indicated; disabling; |
| | limiting self-care ADL. |
| Grade 4 (Life threatening) | Life-threatening consequences; urgent intervention indicated. |
| Grade 5 (Death) | Death. |

ADL: Activities of Daily Living

If the adverse event returns to Grade 1 or baseline, the ripretinib dose should be re-escalated. Efforts must be made to re-escalate the dose to the dose level at which the adverse event occurred. If the dose level is reduced to the first dose reduction level and the adverse event returns to Grade 1 or baseline, the participant may be restarted at the starting dose level. If a participant has 2 sequential dose reductions and the adverse event returns to Grade 1 or baseline at the second dose reduction level, the participant may be re-started at the first dose reduction level and must remain at this dose level for 1 cycle without interruption before escalating to the starting dose level.

If the adverse event leading to ripretinib dose modification does not return to Grade 1 or baseline within 1 cycle (42 days), then ripretinib must be discontinued, unless the event is considered not clinically significant by the Investigator, in which case the possibility of restarting the participant at a reduced dose level may be made after consultation with the Sponsor.

### Example 3. Results From a Bridging Study of Second-Line Ripretinib in the Treatment of GIST

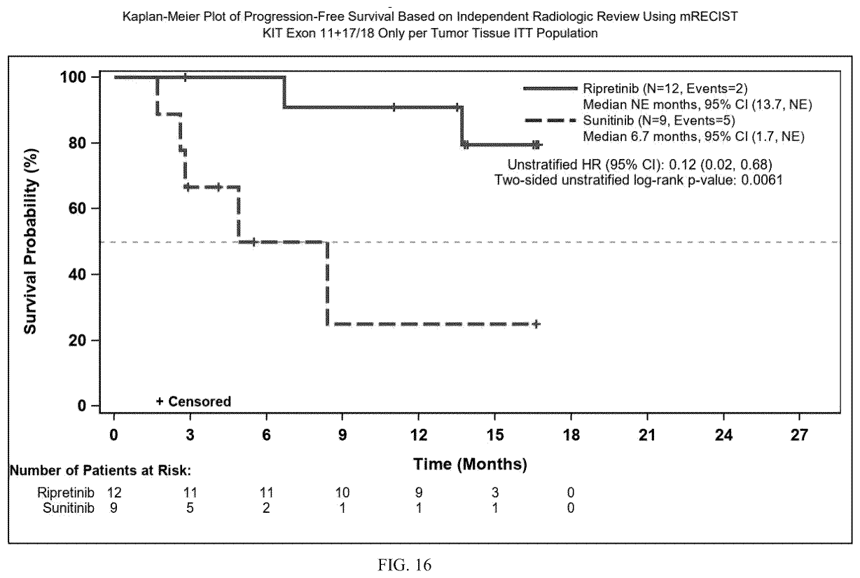

Results from a bridging study, with respect to the study of Example 1 above, of second-line administration of 150 mg once daily ripretinib, as compared to administration of 50 mg once daily sunitinib, in gastrointestinal stromal tumors (GIST) patients in China were obtained. In the study, 21 patients were determined to have mutations at KIT exon 11 and exon 17/18 per the tumor tissue ITT population. Progression-free survival (PFS) was determined with independent radiologic review using mRECIST. A Kaplan-Meier plot of PFS is provided as FIG. 16. The data show that the median PFS in the sunitinib treatment arm was 6.7 months, while PFS in the ripretinib treatment arm was not reached (NE: not evaluable).

### Example 4. Outcomes in Patients With Advanced Gastrointestinal Stromal Tumor who did not Have Baseline ctDNA Detected in a Clinical Study With Ripretinib

In the study of Example 1 described above, efficacy outcomes in patients with advanced gastrointestinal stromal tumors who had ctDNA detected as compared to patients that did not have detectable ctDNA were studied, where ctDNA-D is defined as a sample successfully analyzed with at least one somatic alteration detected (SNV or INDEL). ctDNA was detected in 280/362 patients (77.3%) (ctDNA-D), whereas 82/362 patients (22.7%) did not have detectable ctDNA (ctDNA-ND). Among patients with ctDNA-ND, 40 patients received ripretinib while 42 received sunitinib. Among patients with ctDNA-D, 135 patients received ripretinib and 145 received sunitinib.

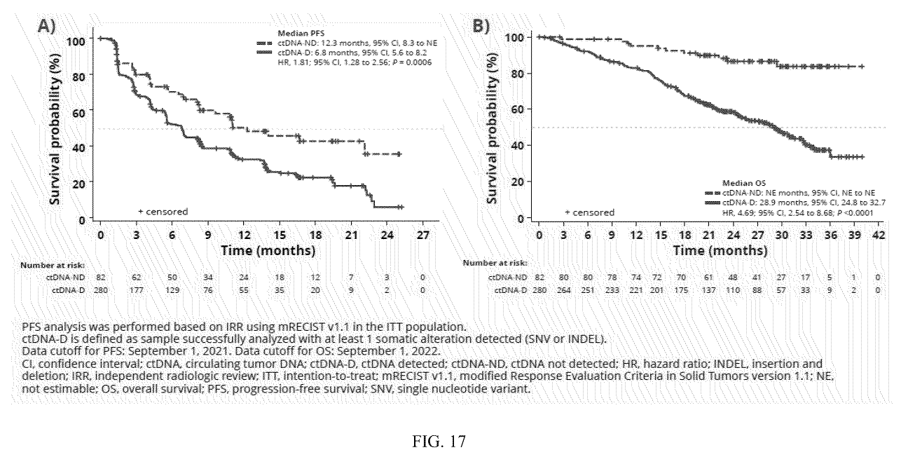

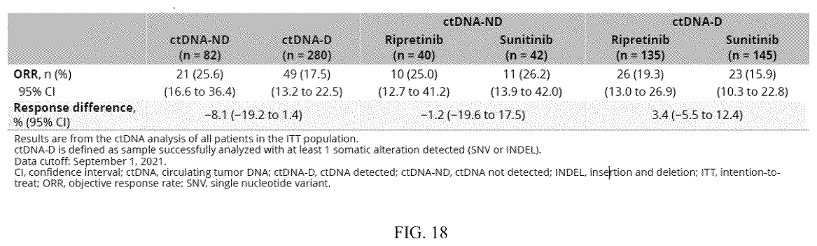

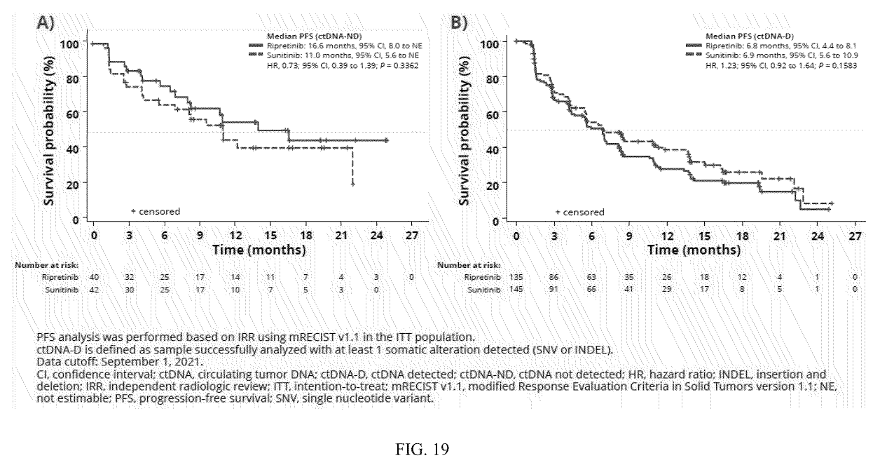

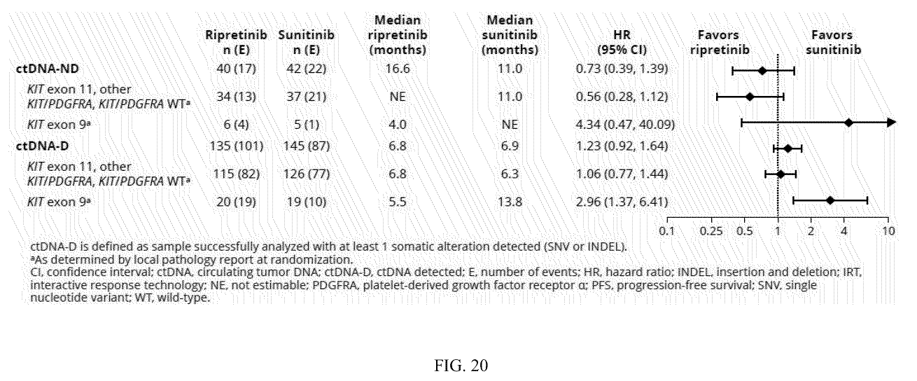

FIG. 17 shows a Kaplan-Meier analysis of PFS (A) and OS (B) for patients with ctDNA-ND versus ctDNA-D. FIG. 18 shows ORR in patients with ctDNA-ND versus ctDNA-D. FIG. 19 shows a Kaplan-Meier analysis of PFS for patients treated with ripretinib or sunitinib in the ctDNA-ND (A) and ctDNA-D (B) populations. FIG. 20 shows a forest plot of PFS by KIT mutational status as determined by local pathology report at randomization.

### Example 5. Multiple Treatment Comparisons Across Mutational Subgroups

Cox proportional hazards analyses of PFS and OS were conducted to examine the interaction of treatment arm and mutational subgroup with or without adjustment for baseline characteristics in the study of Example 1.

With respect to PFS, a Cox model with the interaction effect was used and the Bonferroni adjustment was applied. Interaction analysis between treatment arm and mutational subgroup for PFS indicated that the hazard ratio (HR) values were different across subgroups. The treatment effect was nominally significant in the KIT exon 11+13/14 and KIT exon 11+17/18 populations before and after performing the Bonferroni correction. This interaction analysis also showed nominal significance after adjustment for age, sex, and race; females had a significantly lower risk of disease progression or death compared with males, irrespective of treatment or mutation subgroup. A summary of the results are presented in Table 2 below. The results were robust when accounting for multiple treatment comparisons across mutational subgroups.

TABLE 2
Without adjustment for age, sex, and race

| | | Wald Chi- | Nominal |
|---|---|---|---|
| Effect | DF | Square | P-value |
| Treatment | 1 | 0.0023 | 0.9614 |
| KIT mutational group | 5 | 27.5846 | <0.0001 |
| Treatment by KIT mutational group | 5 | 41.0369 | <0.0001 |
| interaction | | | |

| | | | | Bonferroni- |
|---|---|---|---|---|
| Ripretinib versus sunitinib | | Adjusted | Nominal | adjusted |
| N = 221 | HR | 95% CI | P-value | P-value |
| KIT exon 11 + 13/14 only | 3.41 | 1.17 to 9.94 | 0.0025 | 0.0150 |
| (n = 41) | | | | |
| KIT exon 11 + 17/18 only | 0.22 | 0.09 to 0.52 | <0.0001 | <0.0001 |
| (n = 52) | | | | |
| KIT exon 11 + 13/14 + 17/18 | 0.98 | 0.27 to 3.57 | 0.9744 | 0.9999 |
| only (n = 22) | | | | |
| KIT exon 11 only | 2.53 | 0.84 to 7.65 | 0.0266 | 0.1593 |
| (n = 41) | | | | |
| Any KIT exon 9 | 2.88 | 0.95 to 8.75 | 0.0122 | 0.0735 |
| (n = 36) | | | | |
| Other KIT/PDGFRA | 1.02 | 0.28 to 3.68 | 0.9614 | 0.9999 |
| (n = 29) | | | | |

With adjustment for age, sex, and race

| | | Wald Chi- | Nominal |
|---|---|---|---|
| Effect | DF | Square | P-value |
| Treatment | 1 | 0.0030 | 0.9560 |
| KIT mutational group | 5 | 27.9119 | <0.0001 |
| Treatment by KIT mutational group | 5 | 41.4213 | <0.0001 |
| interaction | | | |
| Age | 1 | 0.9041 | 0.3417 |
| Sex | 1 | 4.3092 | 0.0379 |
| Race | 1 | 0.6829 | 0.4086 |

| | | | | Bonferroni- |
|---|---|---|---|---|
| Ripretinib versus sunitinib | | Adjusted | Nominal | adjusted |
| N = 221 | HR | 95% CI | P-value | P-value |
| KIT exon 11 + 13/14 only | 3.27 | 1.12 to 9.56 | 0.0036 | 0.0216 |
| (n = 41) | | | | |
| KIT exon 11 + 17/18 only | 0.21 | 0.09 to 0.50 | <0.0001 | <0.0001 |
| (n = 52) | | | | |
| KIT exon 11 + 13/14 + 17/18 | 0.96 | 0.26 to 3.53 | 0.9317 | 0.9999 |
| only (n = 22) | | | | |
| KIT exon 11 only | 2.54 | 0.83 to 7.78 | 0.0278 | 0.1670 |
| (n = 41) | | | | |
| Any KIT exon 9 | 3.07 | 1.00 to 9.37 | 0.0082 | 0.0490 |
| (n = 36) | | | | |
| Other KIT/PDGFRA | 0.97 | 0.27 to 3.51 | 0.9560 | 0.9999 |
| (n = 29) | | | | |

Abbreviations: CI, confidence interval; DF, degree of freedom; HR, hazard ratio; PDGFRA, platelet-derived growth factor receptor α; PFS, progression-free survival.

With respect to OS, a Cox model with the interaction effect was used and the Bonferroni adjustment was applied. Interaction analysis between treatment arm and mutational subgroup for OS indicated that the hazard ratio (HR) values were different across subgroups. The treatment effect was nominally significant in the KIT exon 11+17/18 population before and after performing the Bonferroni correction, but not in the KIT exon 11+13/14 population. This analysis also showed nominal significance after adjustment for age, sex, and race. Females showed a trend towards a lower risk of death compared with males and there was a trend towards a higher risk of death in older patients, irrespective of treatment or mutational subgroup. A summary of the results are presented in Table 3 below. The results were robust when accounting for multiple treatment comparisons across mutational subgroups.

TABLE 3
Without adjustment for age, sex, and race

| | | Wald Chi- | Nominal |
|---|---|---|---|
| Effect | DF | Square | P-value |
| Treatment | 1 | 0.2138 | 0.6438 |
| KIT mutational group | 5 | 7.2678 | 0.2015 |
| Treatment by KIT mutational group | 5 | 13.6636 | 0.0179 |
| interaction | | | |

| | | | | Bonferroni- |
|---|---|---|---|---|
| Ripretinib versus sunitinib | | Adjusted | Nominal | adjusted |
| N = 221 | HR | 95% CI | P-value | P-value |
| KIT exon 11 + 13/14 only | 1.88 | 0.57 to 6.14 | 0.1620 | 0.9721 |
| (n = 41) | | | | |
| KIT exon 11 + 17/18 only | 0.32 | 0.11 to 0.95 | 0.0058 | 0.0350 |
| (n = 52) | | | | |
| KIT exon 11 + 13/14 + 17/18 only | 2.15 | 0.61 to 7.60 | 0.1092 | 0.6554 |
| (n = 22) | | | | |
| KIT exon 11 only | 1.64 | 0.55 to 4.91 | 0.2311 | 0.9999 |
| (n = 41) | | | | |
| Any KIT exon 9 | 1.32 | 0.33 to 5.29 | 0.6008 | 0.9999 |
| (n = 36) | | | | |
| Other KIT/PDGFRA | 0.78 | 0.18 to 3.30 | 0.6438 | 0.9999 |
| (n = 29) | | | | |

With adjustment for age, sex, and race

| | | Wald Chi- | Nominal |
|---|---|---|---|
| Effect | DF | Square | P-value |
| Treatment | 1 | 0.6738 | 0.4117 |
| KIT mutational group | 5 | 6.8034 | 0.2357 |
| Treatment by KIT mutational group | 5 | 12.3666 | 0.0301 |
| interaction | | | |
| Age | 1 | 3.7948 | 0.0514 |
| Sex | 1 | 3.0417 | 0.0812 |
| Race | 1 | 0.8671 | 0.3518 |

| | | | | Bonferroni- |
|---|---|---|---|---|
| Ripretinib versus sunitinib | | Adjusted | Nominal | adjusted |
| N = 221 | HR | 95% CI | P-value | P-value |
| KIT exon 11 + 13/14 only | 1.91 | 0.58 to 6.26 | 0.1513 | 0.9076 |
| (n = 41) | | | | |
| KIT exon 11 + 17/18 only | 0.34 | 0.11 to 1.03 | 0.0099 | 0.0596 |
| (n = 52) | | | | |
| KIT exon 11 + 13/14 + 17/18 only | 2.09 | 0.58 to 7.56 | 0.1293 | 0.7758 |
| (n = 22) | | | | |
| KIT exon 11 only | 1.55 | 0.50 to 4.83 | 0.3045 | 0.9999 |
| (n = 41) | | | | |
| Any KIT exon 9 | 1.33 | 0.33 to 5.36 | 0.5872 | 0.9999 |
| (n = 36) | | | | |
| Other KIT/PDGFRA | 0.63 | 0.15 to 2.74 | 0.4117 | 0.9999 |
| (n = 29) | | | | |

Abbreviations: CI, confidence interval; DF, degree of freedom; HR, hazard ratio; PDGFRA, platelet-derived growth factor receptor α; PFS, progression-free survival.

What is claimed is:

1. A method of treating a patient suffering from an advanced gastrointestinal stromal tumor, where the patient has a) a KIT exon 17 and/or a KIT exon 18 mutation and b) a KIT exon 11 mutation, comprising:
administering to the patient 150 mg of a compound represented by: 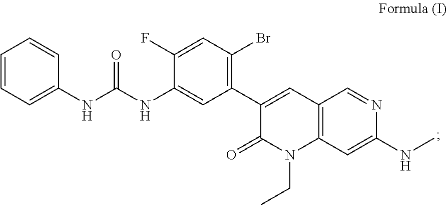 [SMILES: CCn1c(=O)c(-c2cc(NC(=O)Nc3ccccc3)c(F)cc2Br)cc2cnc(NC)cc21]
daily or twice daily, and
where before the administration of the compound, the patient has progressed on or was intolerant to imatinib; and
wherein upon administration of the compound the patient achieves significantly more progression free survival time as determined by mRECIST1.1, and significantly more overall survival time, as compared to a patient with a KIT exon 17 and/or a KIT exon 18 mutation and a KIT exon 11 mutation suffering from an advanced gastrointestinal stromal tumor who has progressed on or was intolerant to imatinib and was then administered 50 mg sunitinib once daily.

2. The method of claim 1, where the patient has a) the KIT exon 17 and/or the KIT exon 18 mutation and b) the KIT exon 11 mutation in circulating tumor DNA.

3. The method of claim 1, wherein the patient being administered the compound does not have a KIT exon 9, or does not have a KIT exon 13 or 14 mutation in circulating tumor DNA.

4. The method of claim 1, wherein the patient being administered the compound does not have a KIT exon 9, and does not have a KIT exon 13 or 14 mutation in circulating tumor DNA.

5. The method of claim 1, wherein the patient being administered the compound has a progression free survival of at least about 14 months as compared to a progression free survival of about 1.5 months for the patient administered sunitinib.

6. The method of claim 1, comprising administering 150 mg of the compound once or twice daily for at least 40 days to the patient.

7. The method of claim 1, comprising administering 150 mg of the compound to the patient once daily.

8. A method of treating a patient suffering from an advanced gastrointestinal stromal tumor who has a KIT exon 17 and/or a KIT exon 18 mutation, does not have KIT exon 13 and/or 14 mutation, and does not have a KIT exon 9 mutation, and where the patient has progressed on or was intolerant to imatinib, comprising administering to the patient 150 mg of a compound represented by: 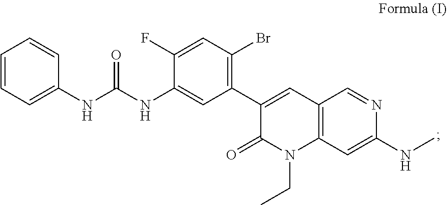 [SMILES: CCn1c(=O)c(-c2cc(NC(=O)Nc3ccccc3)c(F)cc2Br)cc2cnc(NC)cc21]
daily or twice daily.

9. The method of claim 8, wherein the patient has the KIT exon 17 and/or a KIT exon 18 mutation, does not have the KIT exon 13 and/or 14 mutation, and does not have the KIT exon 9 mutation, in circulating tumor DNA.

10. The method of claim 8, wherein the patient's circulating tumor DNA also has a KIT exon 11 mutation.

11. The method of claim 8, comprising administering to the patient 150 mg of the compound daily.

12. A method of treating a patient suffering from an advanced gastrointestinal stromal tumor where the patient has progressed on or was intolerant to imatinib, and the patient's circulating tumor DNA has a KIT exon 17 and/or a KIT exon 18 mutation and does not have a KIT exon 9 mutation, comprising administering to the patient 150 mg of a compound represented by: 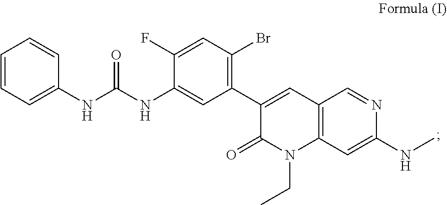 [SMILES: CCn1c(=O)c(-c2cc(NC(=O)Nc3ccccc3)c(F)cc2Br)cc2cnc(NC)cc21]
daily or twice daily.

13. The method of claim 12, comprising administering to the patient 150 mg of the compound daily.

14. A method of treating an advanced gastrointestinal stromal tumor where the patient has progressed on or was intolerant to imatinib in a patient in need thereof, comprising:
selecting a patient having a circulating tumor DNA KIT exon 17 and/or a KIT exon 18 mutation and without one or more of a KIT exon 9, 13, and 14 mutation to obtain a selected patient;
administering to the selected patient 150 mg of a compound represented by: 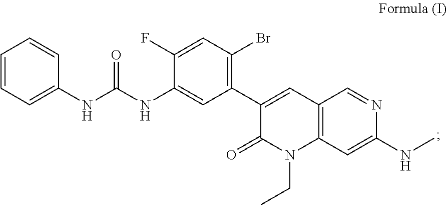 [SMILES: CCn1c(=O)c(-c2cc(NC(=O)Nc3ccccc3)c(F)cc2Br)cc2cnc(NC)cc21]
daily or twice daily;
or if the patient is not a selected patient, administering to the patient one or more different kinase inhibitors.

15. The method of claim 14, wherein if the patient is not a selected patient, the one or more different kinase inhibitors includes sunitinib.

16. The method of claim 14, wherein if the patient is not a selected patient, comprising administering to the patient 50 mg of sunitinib once daily.

17. The method of claim 15, wherein if the patient is not a selected patient and has further progressed at least after the sunitinib administration, further comprising administering to patient 150 mg of the compound daily or twice daily.

18. The method of claim 14, comprising administering to the selected patient 150 mg of the compound once daily.
